(19) United States
(12) Patent Application Publication
(10) Pub. No.: US 2022/0183903 A1
(43) Pub. Date: Jun. 16, 2022

(54) SPECIMEN COLLECTION SWAB ASSEMBLY

(71) Applicant: VECTORNATE KOREA CO., LTD., Jeollanam-do (KR)

(72) Inventors: Sean Taesun YEOM, Woodcliff Lake, NJ (US); Ji Hyun SHIN, Naju-si (KR)

(73) Assignee: VECTORNATE KOREA CO., LTD., Jeollanam-do (KR)

(21) Appl. No.: 17/549,765

(22) Filed: Dec. 13, 2021

Related U.S. Application Data
(60) Provisional application No. 63/125,917, filed on Dec. 15, 2020.

(51) Int. Cl. A61F 13/38; A61B 10/00

(52) U.S. Cl. CPC A61F 13/38; A61B 2090/036; A61B 10/0051

(57) ABSTRACT

A swab includes a stick having a handgrip section and a tip section. The tip section includes a plurality of lobes arranged axially along a longitudinal axis of the stick. More particularly, the lobes include a first lobe located at a distal end of the stick and at least one second lobe axially offset from the first lobe. The diameter of the first lobe is smaller than the diameter of the second lobe. Fibers are attached to the tip section for collecting a specimen from an anatomical cavity of a patient. A stop plate may also be provided for removable attachment to the stick for adjusting the extent to which the tip section can be inserted into the cavity.

### CROSS-REFERENCE TO RELATED APPLICATION

This application claims priority to U.S. Provisional Application Ser. No. 63/125,917 filed Dec. 15, 2020, the entire disclosure of which is incorporated herein by reference for all purposes.

### FIELD OF THE INVENTION

The present invention relates to swabs for collection of specimens from patients.

### BACKGROUND OF THE INVENTION

Various swabs have been developed in the past for collecting biological samples from patients. The specimen collection swab according to the present invention enhances, inter alia, swab functionality and/or operation.

### SUMMARY OF THE INVENTION

In one embodiment, a swab includes a stick having a handgrip section and a tip section. The tip section includes a plurality of lobes arranged axially along a longitudinal axis of the stick. More particularly, the lobes include a first lobe located at a distal end of the stick and at least one second lobe axially offset from the first lobe. The diameter of the first lobe is smaller than the diameter of the second lobe. Fibers are attached to the tip section for collecting a specimen from an anatomical cavity of a patient.

In another embodiment, a swab includes a stick having a swab tip and is provided with a stop plate which can be removably attached to the stick. The stick is provided with a plurality of mounting areas positioned along a longitudinal axis of the stick. The stop plate can be attached to any one of the mounting areas so as to adjust the extent to which the swab tip can be inserted into an anatomical cavity of a patient.

In one embodiment, a swab includes a stick having a handgrip section and a tip section. The tip section includes a plurality of lobes arranged axially along a longitudinal axis of the stick. The plurality of lobes include a first lobe located at a distal end of the stick and a second lobe axially offset from the first lobe. Fibers are attached to the tip section for collecting a specimen from an anatomical cavity of a patient.

In one embodiment, the plurality of swabs further include a third lobe axially offset from the second lobe away from the distal end of the stick.

In one embodiment, the first and second lobes are positioned adjacent to each other, while the second and third lobes are positioned adjacent to each other.

In one embodiment, the first, second and third lobes include first, second and third diameters, respectively. The first diameter of the first lobe is smaller than the second diameter of the second lobe, while the second diameter of the second lobe is smaller than the third diameter of the third lobe.

In one embodiment, the first and second lobes are connected to each other such that a first tapered section is formed between the first and second lobes, while the second and third lobes are connected to each other such that a second tapered section is formed between the second and third lobes.

In one embodiment, the plurality of lobes cooperate to form a snowman shape.

In one embodiment, the first and second lobes are spaced from each other, while the second and third lobes are spaced from each other.

In one embodiment, the first and second lobes are arranged relative to each other such that a first extended area having a reduced diameter is formed between the first and second lobes. In one embodiment, the second and third lobes are arranged relative to each other such that a second extended area having a reduced diameter is formed between the second and third lobes.

In one embodiment, a stop plate is configured to be removably attached to the stick for inhibiting insertion of the tip section into the cavity beyond a predetermined distance.

In one embodiment, the stick includes a plurality of mounting areas provided along the longitudinal axis of the stick. In one embodiment, the stop plate includes a slot and an opening configured for receiving each of the plurality of mounting areas of the stick such that the stop plate can be removably mounted to a selected one of the plurality of mounting areas of the stick.

In one embodiment, each of the plurality of mounting areas of the stick includes a plurality of protrusions, which are arranged along a circumference of a corresponding one of the plurality of the mounting areas of the stick, and a plurality of indentations, each of which is positioned between an adjacent pair of the plurality of protrusions.

In one embodiment, the stop plate includes a plurality of protrusions arranged on the stop plate along at least a portion of a circumference of the opening. In one embodiment, when one of the plurality of mounting areas of the stick is inserted into the opening, each of the plurality of protrusions of the stop plate is positioned in a corresponding one of the plurality of indentations of the one of the plurality of mounting areas.

In one embodiment, the opening is located adjacent a center of the stop plate. The slot communicates with the opening so as to allow passage of each of the plurality of mounting areas of the stick into the opening.

In another embodiment, a swab assembly comprising a swab having a stick, which includes a handgrip section and a tip section, and fibers, which are attached to the tip section for collecting a specimen from an anatomical cavity of a patient. The swab assembly also includes a stop plate configured to be removably attached to the stick at a preselected location for inhibiting insertion of the tip section into the cavity beyond a distance determined by the preselected location of the stop plate on the stick.

In one embodiment, the stick of the swab assembly includes a plurality of mounting areas provided along a longitudinal axis of the stick between the tip section and the handgrip section. In one embodiment, the stop plate of the swab assembly includes a slot and an opening configured for receiving each of the plurality of mounting areas of the stick such that the stop plate can be removably mounted to a selected one of the plurality of mounting areas of the stick.

In one embodiment, the plurality of mounting areas of the stick of the swab assembly includes a plurality of protrusions, which are arranged along at a circumference of a corresponding one of the plurality of the mounting areas of the stick, and a plurality of indentations, each of which is positioned between an adjacent pair of the plurality of protrusions.

In one embodiment, the stop plate of the swab assembly includes a plurality of protrusions arranged on the stop plate along at least a portion of a circumference of the opening. In one embodiment, when one of the plurality of mounting areas of the stick is inserted into the opening, each of the plurality of protrusions of the stop plate is positioned in a corresponding one of the plurality of indentations of the one of the plurality of mounting areas.

In another embodiment, a method of using a swab is provided. The swab has a stick, which includes a handgrip section and a tip section, and fibers, which are attached to the tip section. The method includes the steps of removably attaching a stop plate to the stick at a predetermined location; and inserting the tip section into an anatomical cavity of a patient for collecting a specimen from the patient, whereby the distance by which the tip section can be inserted into the cavity is limited by the stop plate.

In one embodiment, the stick of the method includes a plurality of mounting areas provided along a longitudinal axis of the stick between the tip section and the handgrip section. The stop plate includes a slot and an opening configured for receiving each of the plurality of mounting areas of the stick such that the stop plate can be removably mounted to a selected one of the plurality of mounting areas of the stick.

In one embodiment, each of the plurality of mounting areas of the stick of the method includes a plurality of protrusions, which are arranged along at a circumference of a corresponding one of the plurality of the mounting areas of the stick, and a plurality of indentations, each of which is positioned between an adjacent pair of the plurality of protrusions. The stop plate of the method includes a plurality of protrusions arranged on the stop plate along at least a portion of a circumference of the opening. When one of the plurality of mounting areas of the stick is inserted into the opening, each of the plurality of protrusions of the stop plate is positioned in a corresponding one of the plurality of indentations of the one of the plurality of mounting areas.

### BRIEF DESCRIPTION OF THE DRAWINGS

In the drawing figures, which are not to scale, and where like reference numerals indicate like elements throughout the several views:

FIG. 1 is a perspective view of a swab assembly constructed in accordance with an embodiment, the swab assembly including a swab and a stop plate;

FIG. 2 is a plan view of the swab shown in FIG. 1;

FIG. 3 is an enlarged view of a tip section of the swab shown in FIG. 2;

FIG. 4 is a cross-sectional view, taken along section line 4-4 and looking in the direction of the arrows, of the swab shown in FIG. 3;

FIG. 5 is a perspective view of the stop plate shown in FIG. 1;

FIG. 6 is a plan view of the stop plate shown in FIG. 5;

FIG. 6A is an enlarged view of an area of the stop plate shown in FIG. 6;

FIG. 7 is a view similar to FIG. 6A, except that the swab is engaged with the stop plate;

FIG. 8 is a perspective view of a swab constructed in accordance with an embodiment;

FIG. 9 is an enlarged perspective view of a tip section of the swab shown in FIG. 8;

FIG. 10 is a perspective view of a swab constructed in accordance with an embodiment; and

FIG. 11 is an enlarged perspective view of a tip section of the swab shown in FIG. 10.

### DESCRIPTION OF EXEMPLARY EMBODIMENTS

Embodiments are now discussed in more detail referring to the drawings that accompany the present application. In the accompanying drawings, like and/or corresponding elements are referred to by like reference numbers.

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

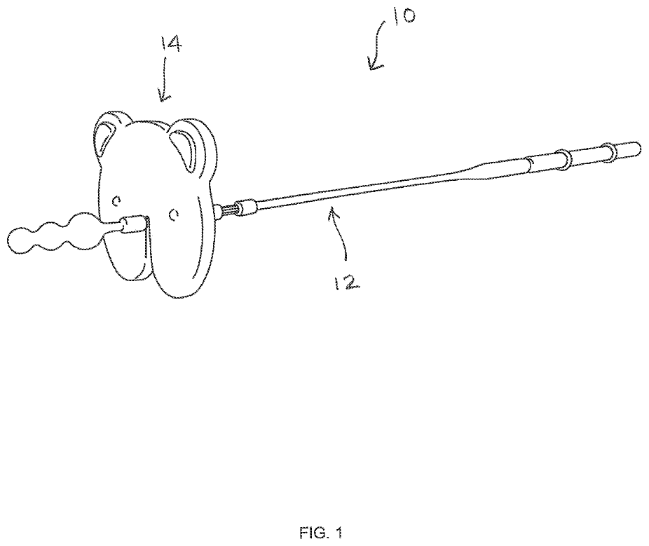

FIG. 1 illustrates a swab assembly 10 constructed in accordance with an embodiment. More particularly, the swab assembly 10 includes a swab 12 for insertion into an anatomical cavity of a patient (e.g., a nasal cavity) in order to collect a specimen from the patient. In one embodiment, the swab assembly 10 is equipped with a stop plate 14. The stop plate 14 can be removably attached to one of a plurality of locations provided on the swab 12. The position of the stop plate 14 on the swab 12 controls the extent to which the swab can be inserted into the cavity. The construction and operation of the swab 12 and the stop plate 14 will be discussed in greater detail below.

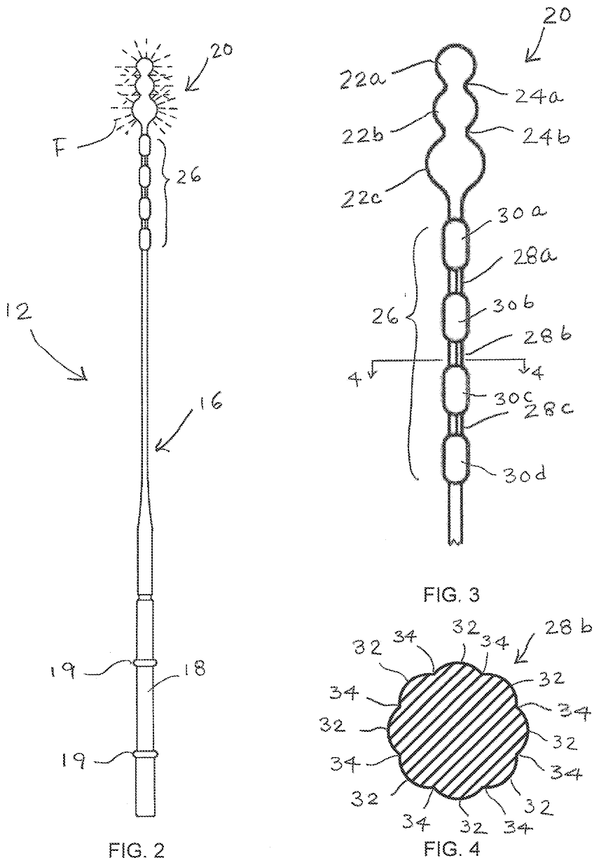

With reference to FIG. 2, the swab 12 has a stick 16 having a handgrip section 18 located at a proximal end of the stick 16 and configured to be gripped with a user, such as medical personnel (e.g., a doctor or a nurse). In one embodiment, the handgrip section 18 includes a plurality of ridges 19 configured to provide a firm grip to the user when collecting samples from a patent. In one embodiment, the ridges 19 are configured to provide a “wider” handle for enhanced control of the swab 12 so as to lessen the pain and/or discomfort that it exerts on the patient, e.g., the patient's nasopharynx and oropharynx.

The stick 16 also includes a tip section 20 located at a distal end of the stick 16 opposite the handgrip section 18. The tip section 20 is sized and shaped for insertion into an anatomical cavity of a patient for the purpose of collecting a specimen from the cavity. In one embodiment, the tip section 20 is configured for insertion into a nasal cavity for collecting a nasal fluid sample from one or more areas in the nasal cavity (e.g., from one or more of the inferior turbinate, middle turbinate, superior turbinate areas). In other embodiments, the tip section 20 is designed for collecting samples from other parts of the patient's body, such as the throat. In one embodiment, the tip section 20 and/or the stick 16 are made to be flexible such that the swab 12 can conform to the contours of a cavity into which it is inserted. The stick 16 can be made from any suitable material, such as plastic, thermoplastic, nylon fibers, nylon, polyester, polyester fibers, etc.

In one embodiment, the tip section 20 of the stick 16 includes a plurality of lobes 22a, 22b, 22c (see FIGS. 2 and 3) which are serially arranged in a linear fashion along a longitudinal axis of the stick 16 and/or the tip section 20. Referring to FIG. 3, the lobe 22a is located at the most distal point of the stick 16 and includes a generally round shape (e.g., a substantially spherical shape), while the lobe 22b is axially offset from the lobe 22a and has a diameter greater than that of the lobe 22a. Similarly, the lobe 22c is axially offset from the lobe 22b and has a diameter greater than that of the lobe 22b. In one embodiment, the lobes 22a, 22b and 22c have radii of about 1.25 mm, about 1.38 mm and about 1.88 mm, respectively. In one embodiment, each adjacent pair of the lobes 22a-22c are connected to (i.e., contiguous with) each other. In one embodiment, the lobes 22a, 22b cooperate to form threrebetween a tapered section 24a having a diameter smaller than the diameters of the lobes, 22a, 22b, while the lobes 22b, 22c cooperate to form therebetween a tapered section 24b having a diameter smaller than the diameters of the lobes 22b, 22c. In one embodiment, the lobes 22a-22c cooperate to form a “snowman” shape, as illustrated in FIGS. 2 and 3. In one embodiment, the distance between the peak (i.e., the laterally expanding portion with the largest diameter) of the lobe 22a and the peak of the lobe 22b is from about 2.75 mm to about 3.25 mm (e.g., about 3.07 mm), while the distance between the peak of the lobe 22b and the peak of the lobe 22c is about from about 3.5 mm to about 4 mm (e.g., about 3.72 mm). In another embodiment, each adjacent pair of the lobes 22a-22c are spaced or separated axially from each other, thereby forming an extended reduced-diameter area between each adjacent pair of the lobes 22a-22c. In other embodiments, the number of the lobes 22a-22c can be more than or less than 3.

The swab 12 also includes a plurality of fibers F (shown in broken line representation in FIG. 2) attached to the tip section 20 of the stick 16. The fibers F can be attached to the tip section 20 in any conventional manner, such as a flocking process. In one embodiment, the overall outer shape of the fibers F generally corresponds to that of the underlying tip section 20 (e.g., a “snowman” shape). In other embodiments, the overall outer shape of the fibers F is different from that of the underlying tip section 20.

Still referring to FIGS. 2 and 3, the stick 16 has a mounting section 26 extending from the tip section 20 for removable attachment of the stop plate 14 to the stick 16. In one embodiment, the mounting section 26 includes a plurality of mounting areas 28a, 28b, 28c, each of which is sized and shaped for engagement with the stop plate 14. In one embodiment, the mounting areas 28a-28c are arranged axially along the longitudinal axis of the stick 16. In another embodiment, each of the mounting areas 28a-28c is located between an adjacent pair of retainers 30a, 30b, 30c, 30d (i.e., each adjacent pair of the mounting areas 28a-28c are separated by a corresponding one of the retainers 30a-30d). Each of the retainers 30a-30d has a diameter larger than the diameter of each of the mounting areas 28a-28c for restricting axial movement of the stop plate 14 once the stop plate 14 is attached to a selected one of the mounting areas 28a-28c.

With reference to FIGS. 3 and 4, in one embodiment, each of the mounting areas 28a-28c includes a cross-section having a scalloped shape. More particularly, each of the mounting areas 28a-28c includes one or more protrusions 32 arranged along the circumference of the corresponding one of the mounting areas 28a-28c. In one embodiment, each of the protrusions 32 has a round, concave shape. Between each adjacent pair of the protrusions 32, a depression or indentation 34 is formed for purposes to be discussed hereinbelow.

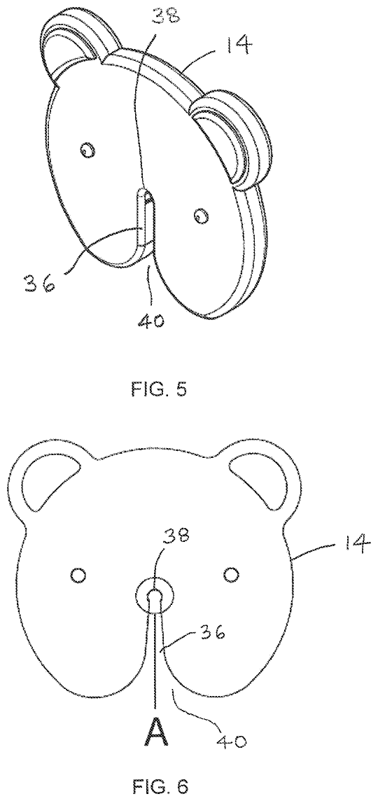

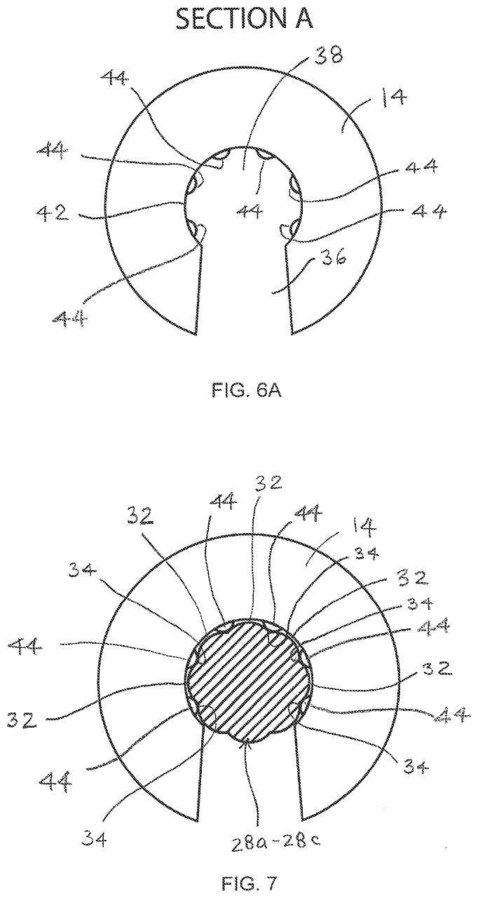

Now referring to FIGS. 5 and 6, the stop plate 14 includes a slot 36 extending from one side of the stop plate 14 and terminating at an opening 38. The slot 36 includes an outwardly flaring mouth 40 for receiving the stick 16. The opening 38 is located at or adjacent a center of the stop plate 14 and is defined by an interior surface 42 of the stop plate 14 (see FIG. 6A). In some embodiments, the interior surface 42 has a generally circular shape and includes protrusions 44 arranged radially on the interior surface 42. In one embodiment, each of the protrusions 44 is sized and shaped so as to be received in one of the indentations 34 formed in the mounting areas 28a-28c of the stick 16 (see FIG. 7) when the stop plate 14 is mounted to one of the mounting areas 28a-28c of the stick 16.

As discussed above, the swab 12 is used for collecting a sample from an anatomical cavity (e.g., a nasal cavity) of a patient. The amount of insertion of the swab 12 into the cavity is determined by selectively attaching the stop plate 14 to one of the mounting areas 28a-28c of the stick 16 (see, e.g., FIG. 1). More particularly, the stop plate 14 is configured to prevent the tip portion 20 of the swab 12 from being inserted into the cavity beyond a predetermined extent (i.e., the distance between the distal end of the stick 16 and the point at which the stop plate 14 is attached to the stick 16). By selectively attaching the stop plate 14 to one of the mounting areas 28a-28c, the distance by which the tip portion 20 is inserted into the cavity can be adjusted.

In one embodiment, the mounting areas 28a-28c are configured so as to make the swab 12 suitable for substantially universal use for patients of all ages (e.g., adults, children and babies). For instance, when obtaining a sample from an adult, the swab 12 can be used without installing the stop plate 14 on the stick 16 or installing it to the mounting area 28c. Alternatively, when the swab 12 is used for obtaining a sample from a child, the stop plate 12 may be installed to the mounting area 28a or the mounting area 28b, depending on the child's size and age. In one embodiment, the lengths from the distal end of the tip section 20 to the mounting areas 28a-28c are about 15.3 mm, about 20.1 mm and 25.5 mm, respectively. In other embodiments, different lengths may be utilized, and/or additional mounting areas may be added to provide additional mounting locations for the stop plate 14.

In one embodiment, the stop plate 14 is removably attached to a selected one of the mounting areas 28a-28c (e.g., the mounting area 28a in the embodiment shown in FIG. 1) by inserting the selected mounting area 28a-28c into the opening 38 of the stop plate 14 through the open mouth 40 and the slot 36. In one embodiment, each of the mounting areas 28a-28c is configured to be snap-fitted into the opening 38 of the stop plate 14 such that the stop plate 14 remains on the swab 12 until its removal. In one embodiment, when a selected one of the mounting areas 28a-28c is inserted into the opening 38, each of the protrusions 44 of the stop plate 14 is placed in a corresponding one of the indentations 34 and is retained between a corresponding adjacent pair of the protrusions 32 of the mounting areas 28a-28c, thereby inhibiting the stop plate 14 from rotating relative to the stick 16. The axial movement of the stop plate 14 is also restricted by a corresponding one or adjacent pair of retainers 30a-30d of the stick 16, thereby retaining the stop plate 14 on the selected one of the mounting areas 28a-28c.

In certain embodiments, the stop plate 16 can be removably attached to the stick 16 of the swab 14 in a number of different ways. For instance, the opening 38 of the stop plate 14 and each of the retaining areas 28a-28c of the swab 12 can be provided with mating or matching polygonal shapes such that each of the retaining areas 28a-28c can be snap-fitted into the opening 38 of the stop plate 14 and such that the stop plate 14 is inhibited from rotating relative to the stick 16. Alternatively, a magnetic mechanism may be utilized for attaching the stop plate 16 to the stick 16.

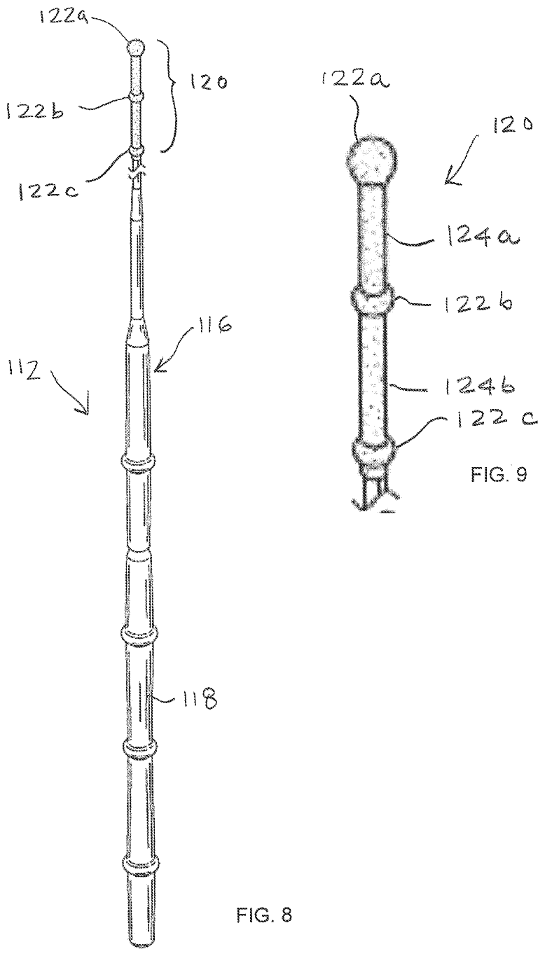

FIGS. 8 and 9 illustrate a swab constructed in accordance with an alternate embodiment of the present invention. Unless stated otherwise, the swab illustrated in FIGS. 8 and 9 is constructed and used in substantially the same manner as those discussed above in connection with FIGS. 1-7. Elements illustrated in FIGS. 8 and 9, which correspond substantially in construction and/or operation to the elements shown in FIGS. 1-7, will be designated with corresponding reference numerals increased by 100.

With reference to FIGS. 8 and 9, there is shown a swab 112 constructed in accordance with one embodiment. More particularly, the swab 112 has a stick 116 having a handgrip section 118 located at a proximal end of the stick 116 and configured to be gripped with a user, such as medical personnel (e.g., a doctor or a nurse). The stick 116 also includes a tip section 120 located at a distal end of the stick 116 opposite the handgrip section 118. The tip section 120 is sized and shaped for insertion into an anatomical cavity of a patient for the purpose of collecting a specimen from the cavity. In one embodiment, the tip section 120 is configured for insertion into a nasal cavity for collecting a nasal fluid sample from one or more areas in the nasal cavity. In other embodiments, the tip section 120 is designed for collecting samples from other parts of the patient's body, such as the throat. In one embodiment, the tip section 120 and/or the stick 116 are made to be flexible such that the swab 112 can conform to the contours of a cavity into which it is inserted.

In one embodiment, the tip section 120 of the stick 116 includes a plurality of lobes 122a, 122b, 122c (see FIGS. 8 and 9) which are arranged in a linear fashion along a longitudinal axis of the stick 116 and/or the tip section 120. Referring to FIG. 9, the lobe 122a is located at the most distal point of the stick 116 and includes a generally round shape (e.g., a substantially spherical shape), while the lobe 122b is axially offset from the lobe 122a and has a diameter substantially equal to that of the lobe 122a in accordance with one embodiment. Similarly, the lobe 122c is axially offset from the lobe 122b and has a diameter substantially equal to that of the lobe 122b. Each of the lobes 122a-122c expands radially outwardly from the tip section 120. In one embodiment, the lobes 122a-122c have outer diameters different from each other. For instance, the diameter of the lobe 122a can be larger or smaller than those of the lobe 122b and/or the lobe 122c. In another embodiment, each adjacent pair of the lobes 122a-122c are spaced or separated axially from each other, thereby forming an extended area 124a, 124b having a reduced diameter between each adjacent pair of the lobes 122a-122c. In a further embodiment, each adjacent pair of the lobes 122a-122c are connected to (i.e., contiguous with) each other (similar to the embodiment shown in FIG. 1), the lobes 122a, 122b and the lobes 122b, 122c cooperating to form threrebetween tapered sections which have diameters smaller than the outer diameters of the lobes 122a, 122b and the lobes 122b, 122c. In other embodiments, the number of the lobes 122a-122c can be more than or less than 3.

In one embodiment, the swab 112 may be provided with a mounting section similar to the mounting section 26 of the swab 12 shown in FIGS. 1-4, as well as a stopper plate similar to the stopper plate 14 shown in FIGS. 1 and 5-6A.

In one embodiment, the swab 112 includes a plurality of fibers attached to the tip section 120 of the stick 116. The fibers can be attached to the tip section 120 in any conventional manner, such as a flocking process. In one embodiment, the overall outer shape of the fibers generally corresponds to that of the underlying tip section 120. In other embodiments, the overall outer shape of the fibers is different from that of the underlying tip section 120.

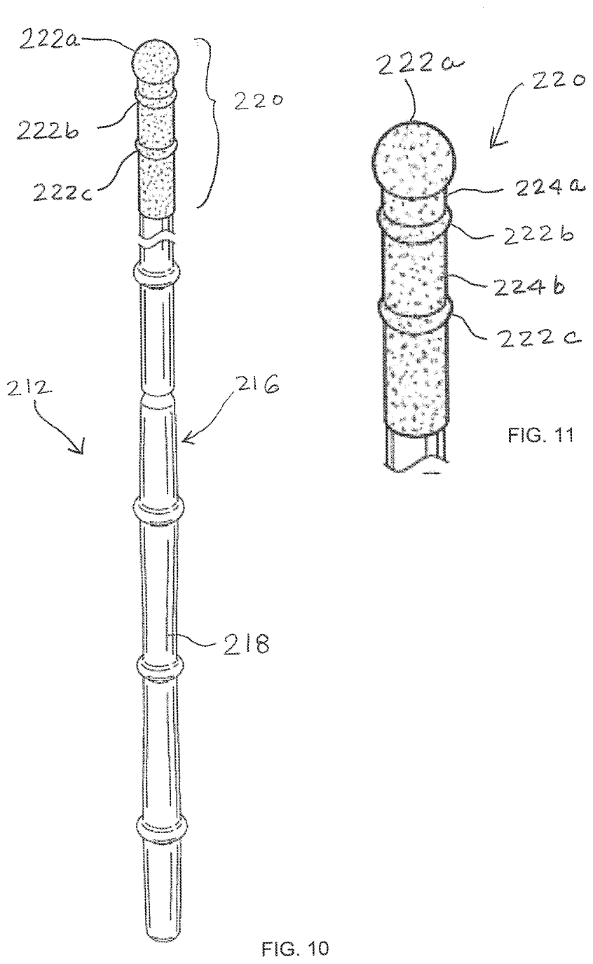

FIGS. 10 and 11 illustrate a swab constructed in accordance with an alternate embodiment of the present invention. Unless stated otherwise, the swab illustrated in FIGS. 10 and 11 is constructed and used in substantially the same manner as those discussed above in connection with FIGS. 1-9. Elements illustrated in FIGS. 10 and 11, which correspond substantially in construction and/or operation to the elements shown in FIGS. 1-7, will be designated with corresponding reference numerals increased by 200.

With reference to FIGS. 10 and 11, there is shown a swab 212 constructed in accordance with one embodiment. More particularly, the swab 212 has a stick 216 having a handgrip section 218 located at a proximal end of the stick 216 and configured to be gripped with a user, such as medical personnel (e.g., a doctor or a nurse). The stick 216 also includes a tip section 220 located at a distal end of the stick 216 opposite the handgrip section 218. The tip section 220 is sized and shaped for insertion into an anatomical cavity of a patient for the purpose of collecting a specimen from the cavity. In one embodiment, the tip section 220 is configured for insertion into a nasal cavity for collecting a nasal fluid sample from one or more areas in the nasal cavity. In other embodiments, the tip section 220 is designed for collecting samples from other parts of the patient's body, such as the throat. In one embodiment, the tip section 220 and/or the stick 216 are made to be flexible such that the swab 212 can conform to the contours of a cavity into which it is inserted.

In one embodiment, the tip section 220 of the stick 216 includes a plurality of lobes 222a, 222b, 222c (see FIGS. 10 and 11) which are arranged in a linear fashion along a longitudinal axis of the stick 216 and/or the tip section 220. Referring to FIG. 9, the lobe 222a is located at the most distal point of the stick 216 and includes a generally round shape (e.g., a substantially spherical shape), while the lobe 222b is axially offset from the lobe 222a and has a diameter substantially equal to that of the lobe 222a. Similarly, the lobe 222c is axially offset from the lobe 222b and has a diameter substantially equal to that of the lobe 222b. Each of the lobes 222a-222c expands radially outwardly from the tip section 120. In one embodiment, each adjacent pair of the lobes 222a-222c are spaced or separated axially from each other, thereby forming an extended area 224a, 224b having a reduced diameter between each adjacent pair of the lobes 222a-222c. In one embodiment, the lobes 222a-222c have outer diameters different from each other. For instance, the diameter of the lobe 222a can be larger or smaller than those of the lobe 222b and/or the lobe 222c. In another embodiment, each adjacent pair of the lobes 222a-222c are connected to (i.e., contiguous with) each other, the lobes 222a, 222b and the lobes 222b, 222c cooperating to form threrebetween tapered sections having diameters smaller than the outer diameters of the lobes 222a, 222b and the lobes 222b, 222c. In other embodiments, the number of the lobes 222a-222c can be more than or less than 3.

In one embodiment, the swab 212 may be provided with a mounting section similar to the mounting section 26 of the swab 12 shown in FIGS. 1-4, as well as a stopper plate similar to the stopper plate 14 shown in FIGS. 1 and 5-6A.

In one embodiment, the swab 212 includes a plurality of fibers attached to the tip section 220 of the stick 216. The fibers can be attached to the tip section 220 in any conventional manner, such as a flocking process. In one embodiment, the overall outer shape of the fibers generally corresponds to that of the underlying tip section 220. In other embodiments, the overall outer shape of the fibers is different from that of the underlying tip section 220.

It will be understood that the embodiments described herein are merely exemplary and that a person skilled in the art may make many variations and modifications without departing from the spirit and scope of the invention. All such variations and modifications are intended to be included within the scope of the invention as defined by the appended claims.

We claim:

1. A swab comprising a stick having a handgrip section and a tip section, said tip section including a plurality of lobes arranged axially along a longitudinal axis of said stick, said plurality of lobes including a first lobe located at a distal end of said stick and a second lobe axially offset from said first lobe; and fibers attached to said tip section for collecting a specimen from an anatomical cavity of a patient.

2. The swab of claim 1, wherein said plurality of swabs further include a third lobe axially offset from said second lobe away from said distal end of said stick.

3. The swab of claim 2, wherein said first and second lobes are positioned adjacent to each other; and wherein said second and third lobes are positioned adjacent to each other.

4. The swab of claim 3, wherein said first, second and third lobes include first, second and third diameters, respectively, said first diameter of said first lobe being smaller than said second diameter of said second lobe, said second diameter of said second lobe being smaller than said third diameter of said third lobe.

5. The swab of claim 4, wherein said first and second lobes are connected to each other such that a first tapered section is formed between said first and second lobes; and wherein said second and third lobes are connected to each other such that a second tapered section is formed between said second and third lobes.

6. The swab of claim 5, wherein said plurality of lobes cooperate to form a snowman shape.

7. The swab of claim 2, where said first and second lobes are spaced from each other;
and wherein said second and third lobes are spaced from each other.

8. The swab of claim 7, wherein said first and second lobes are arranged relative to each other such that a first extended area having a reduced diameter is formed between said first and second lobes; and wherein said second and third lobes are arranged relative to each other such that a second extended area having a reduced diameter is formed between said second and third lobes.

9. The swab of claim 1, further including a stop plate configured to be removably attached to said stick for inhibiting insertion of said tip section into the cavity beyond a predetermined distance.

10. The swab of claim 9, wherein said stick includes a plurality of mounting areas provided along said longitudinal axis of said stick; and wherein said stop plate includes a slot and an opening configured for receiving each of said plurality of mounting areas of said stick such that said stop plate can be removably mounted to a selected one of said plurality of mounting areas of said stick.

11. The swab of claim 10, wherein each of said plurality of mounting areas of said stick includes a plurality of protrusions, which are arranged along a circumference of a corresponding one of said plurality of said mounting areas of said stick, and a plurality of indentations, each of which is positioned between an adjacent pair of said plurality of protrusions.

12. The swab of claim 11, wherein said stop plate includes a plurality of protrusions arranged on said stop plate along at least a portion of a circumference of said opening; and wherein when one of said plurality of mounting areas of said stick is inserted into said opening, each of said plurality of protrusions of said stop plate is positioned in a corresponding one of said plurality of indentations of said one of said plurality of mounting areas.

13. The swab of claim 12, wherein said opening is located adjacent a center of said stop plate, said slot communicating with said opening so as to allow passage of each of said plurality of mounting areas of said stick into said opening.

14. A swab assembly comprising a swab having a stick, which includes a handgrip section and a tip section, and fibers, which are attached to said tip section for collecting a specimen from an anatomical cavity of a patient; and a stop plate configured to be removably attached to said stick at a preselected location for inhibiting insertion of said tip section into the cavity beyond a distance determined by said preselected location of said stop plate on said stick.

15. The swab assembly of claim 14, wherein said stick includes a plurality of mounting areas provided along a longitudinal axis of said stick between said tip section and said handgrip section; and wherein said stop plate includes a slot and an opening configured for receiving each of said plurality of mounting areas of said stick such that said stop plate can be removably mounted to a selected one of said plurality of mounting areas of said stick.

16. The swab assembly of claim 15, wherein each of said plurality of mounting areas of said stick includes a plurality of protrusions, which are arranged along at a circumference of a corresponding one of said plurality of said mounting areas of said stick, and a plurality of indentations, each of which is positioned between an adjacent pair of said plurality of protrusions.

17. The swab assembly of claim 16, wherein said stop plate includes a plurality of protrusions arranged on said stop plate along at least a portion of a circumference of said opening;
and wherein when one of said plurality of mounting areas of said stick is inserted into said opening, each of said plurality of protrusions of said stop plate is positioned in a corresponding one of said plurality of indentations of said one of said plurality of mounting areas.

18. A method of using a swab having a stick, which includes a handgrip section and a tip section, and fibers, which are attached to the tip section, said method comprising the steps of:
removably attaching a stop plate to the stick at a predetermined location; and inserting the tip section into an anatomical cavity of a patient for collecting a specimen from the patient, whereby the distance by which the tip section can be inserted into the cavity is limited by said stop plate.

19. The method of claim 18, wherein the stick includes a plurality of mounting areas provided along a longitudinal axis of the stick between the tip section and the handgrip section;
wherein the stop plate includes a slot and an opening configured for receiving each of the plurality of mounting areas of the stick such that the stop plate can be removably mounted to a selected one of the plurality of mounting areas of the stick.

20. The method of claim 19, wherein each of the plurality of mounting areas of the stick includes a plurality of protrusions, which are arranged along at a circumference of a corresponding one of the plurality of the mounting areas of the stick, and a plurality of indentations, each of which is positioned between an adjacent pair of the plurality of protrusions; wherein the stop plate includes a plurality of protrusions arranged on the stop plate along at least a portion of a circumference of the opening; and wherein when one of the plurality of mounting areas of the stick is inserted into the opening, each of the plurality of protrusions of the stop plate is positioned in a corresponding one of the plurality of indentations of the one of the plurality of mounting areas.
